(19) United States
(12) Patent Application Publication
(10) Pub. No.: US 2008/0261984 A1
(43) Pub. Date: Oct. 23, 2008

(54) Use of S,S-Reboxetine in the Treatment of Pain

(75) Inventors: Bernadette Hughes, Sandwich (GB); Ian McKenzie, Sandwich (GB); Malcolm John Stoker, Sandwich (GB)

Correspondence Address: PHARMACIA CORPORATION;GLOBAL PATENT DEPARTMENT, POST OFFICE BOX 1027, ST. LOUIS, MO, 63006, US

(73) Assignee: Pfizer Inc., New York, NY (US)

(21) Appl. No.: 11/570,280

(22) PCT Filed: May 27, 2005
(86) PCT No.: PCT/IB05/01904

§ 371 Date: Dec. 8, 2006

Related U.S. Application Data
(60) Provisional application No. 60/588,652, filed on Jul. 16, 2004.
(60) Provisional application No. 60/634,427, filed on Dec. 7, 2004.

(30) Foreign Application Priority Data
Jun. 9, 2004 (GB) 0412878.1
Nov. 12, 2004 (GB) 0425061.9

(51) Int. Cl. A61K 31/5375; A61P 29/00; A61P 25/00

(52) U.S. Cl. 514/239.2

(57) ABSTRACT

Use of (S,S)- or racemic reboxetine or a pharmaceutically acceptable salt thereof in the manufacture of a medicament for the treatment of a pain condition selected from neuropathic pain, nociceptive pain, cancer pain, back pain, inflammatory pain, musculo-skeletal disorders, visceral pain, pain from strains/sprains, post-operative pain, posttraumatic pain, burns, renal colic, acute pain, central nervous system trauma, head pain, and orofacial pain, is disclosed. Use of (S,S)-reboxetine or a pharmaceutically acceptable salt thereof in the manufacture of a medicament for the treatment of pain in a patient refractory to an alpha-2-delta ligand, and use of (S,S)- or racemic reboxetine or a pharmaceutically acceptable salt thereof in the manufacture of a medicament for use as a mental performance or mood enhancer, are also disclosed.

### FIELD OF THE INVENTION

This invention relates to a number of novel pain uses for (S,S)- or racemic reboxetine.

### DESCRIPTION OF THE PRIOR ART

U.S. Pat. No. 4,229,449 discloses the compound (RR, SS)-2-[α-(2-ethoxyphenoxy)benzyl]morpholine and pharmaceutically acceptable salts thereof, which possesses useful antidepressant properties. This compound is also known as racemic reboxetine.

Many organic compounds exist in optically active forms, i.e., they have the ability to rotate the plane of plane-polarized light. In describing an optically active compound the prefixes R and S are used to denote the absolute configuration of the molecule about its chiral center(s). For a given chemical structure, each of a pair of enantiomers are identical except that they are non-superimposable mirror images of one another. A specific stereoisomer may also be referred to as an enantiomer, and a 1:1 mixture of such isomers is often called an enantiomeric, or racemic, mixture.

When two chiral centres exist in one molecule, there are four possible stereoisomers: (R,R), (S,S), (R,S), and (S,R). Of these, (R,R) and (S,S) are an example of a pair of enantiomers (mirror images of each other), which typically share chemical properties and melting points just like any other enantiomeric pair. The mirror images of (R,R) and (S,S) are not, however, superimposable on (R,S) and (S,R). This relationship is called diastereoisomeric.

Chemically, racemic reboxetine has two chiral centres and is defined as the (R,R) and (S,S) enantiomeric pair. Currently, reboxetine is commercially available only as a racemic mixture of enantiomers, (R,R) and (S,S) in a 1:1 ratio, and reference herein to the generic name ‘reboxetine’ refers to this enantiomeric, or racemic, mixture. Reboxetine is commercially sold under the trade names of EDRONAX™, PROLIFT™, VESTRA™, and NOREBOX™.

(S,S)-reboxetine and related compounds are disclosed in GB-A-2167407, incorporated in its entirety by reference.

Reboxetine does not act like most antidepressants. Unlike tricyclic antidepressants, and even selective serotonin reuptake inhibitors (SSRIs), reboxetine is ineffective in the 8-OH-DPAT hypothermia test, indicating that reboxetine is not a SSRI. See Brian E. Leonard, ‘Noradrenaline in basic models of depression.’ European Neuropsychopharmacol, 7 Suppl. 1 pp. S11-6 and S71-3 (April 1997), incorporated herein in its entirety by reference thereto. (S,S)-Reboxetine is a selective norepinephrine reuptake inhibitor, with only marginal serotonin and no dopamine reuptake inhibitory activity. Reboxetine displays no anticholinergic binding activity in different animal models, and is substantially devoid of monoamine oxidase (MAO) inhibitory activity.

It is known that (S,S)-reboxetine possesses greatly improved selectivity for norepinephrine reuptake over serotonin reuptake. Accordingly, WO 01/01973 discloses a method of selectively inhibiting reuptake of norepinephrine, the method comprising the step of administering a therapeutically effective amount of a composition to an individual, the composition comprising a compound having a pharmacological selectivity of serotonin (Kᵢ)/norepinephrine (Kᵢ) of at least about 5000. The document further discloses a number of novel uses of (S,S)- and racemic reboxetine, including in the treatment of chronic pain, peripheral neuropathy, fibromyalgia and other somatoform disorders, and migraine headaches.

There is an important medical need for improved therapies for many types of pain conditions. We have now surprisingly found that (S,S)- and racemic reboxetine may be used in the treatment of further pain conditions in addition to those disclosed above.

An alpha-2-delta receptor ligand is any molecule which binds to any sub-type of the human calcium channel alpha-2-delta sub-unit. The calcium channel alpha-2-delta sub-unit comprises a number of receptor sub-types which have been described in the literature: for example, see N. S. Gee, J. P. Brown, V. U. Dissanayake, J. Offord, R. Thurlow, and G. N. Woodruff, J-Biol-Chem 271 (10):5768-76, 1996, (type 1); Gong, J. Hang, W. Kohler, Z. Li, and T-Z. Su, J. Membr. Biol. 184 (1):35-43, 2001, (types 2 and 3); E. Marais, N. Klugbauer, and F. Hofmann, Mol. Pharmacol. 59 (5):1243-1248, 2001. (types 2 and 3); and N. Qin, S. Yagel, M. L. Momplaisir, E. E. Codd, and M. R. D'Andrea. Mol. Pharmacol. 62 (3):485-496, 2002, (type 4). They may also be known as GABA analogs.

Alpha-2-delta ligands have been described for a number of indications. The best known alpha-2-delta ligand, gabapentin (Neurontin®), 1-(aminomethyl)-cyclohexylacetic acid, was first described in the patent literature in the patent family comprising U.S. Pat. No. 4,024,175. The compound is approved for the treatment of epilepsy and neuropathic pain.

A second alpha-2-delta ligand, pregabalin, (S)-(+)-4-amino-3-(2-methylpropyl)butanoic acid, is described in EP-A-641330 as an anti-convulsant treatment useful in the treatment of epilepsy and in EP-A-934061 for the treatment of pain.

Further alpha-2-delta ligands are described in the following documents.

International Patent Application Publication No. WO-A-01/28978, describes a series of novel bicyclic amino acids, their pharmaceutically acceptable salts, and their prodrugs of formula:

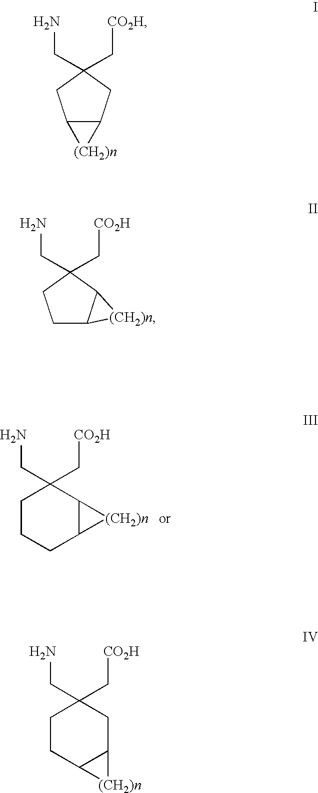

wherein n is an integer of from 1 to 4. Where there are stereocentres, each center may be independently R or S, preferred compounds being those of Formulae I-IV above in which n is an integer of from 2 to 4.

International Patent Application Publication Number WO-A-02/85839 describes alpha-2-delta ligands of the following formulae:

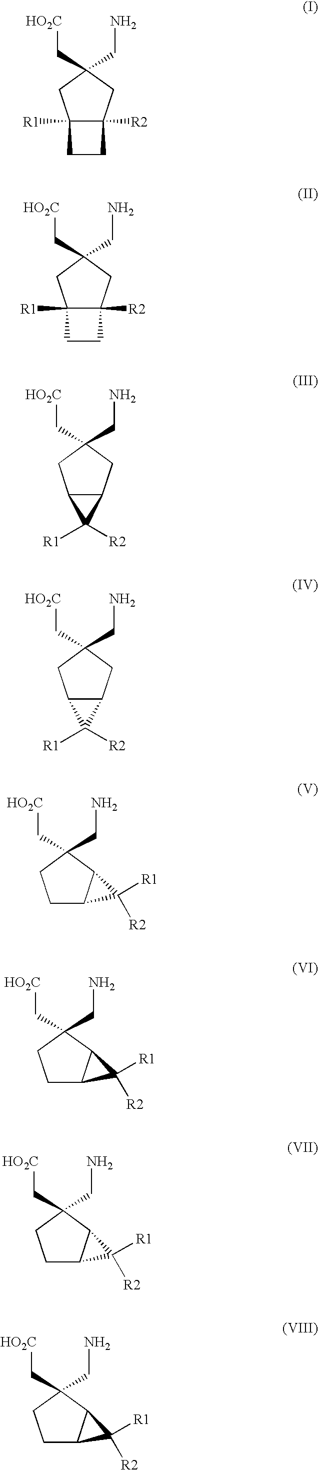

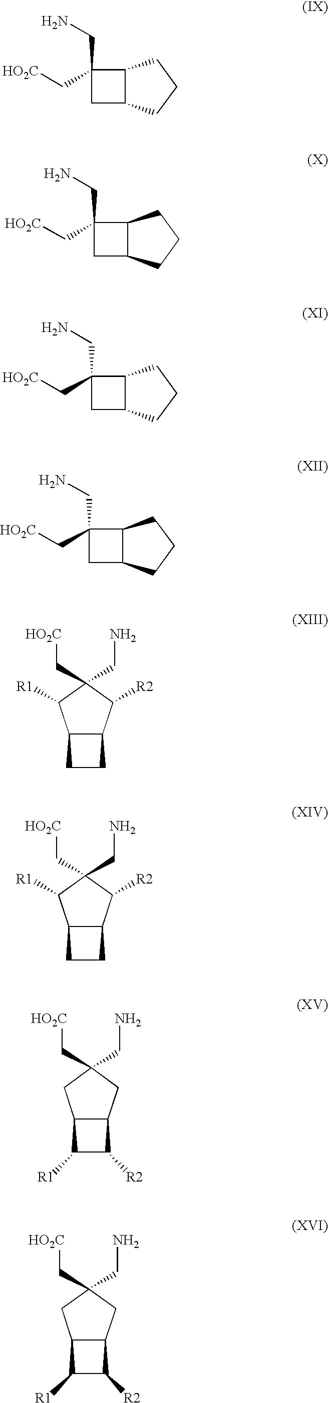

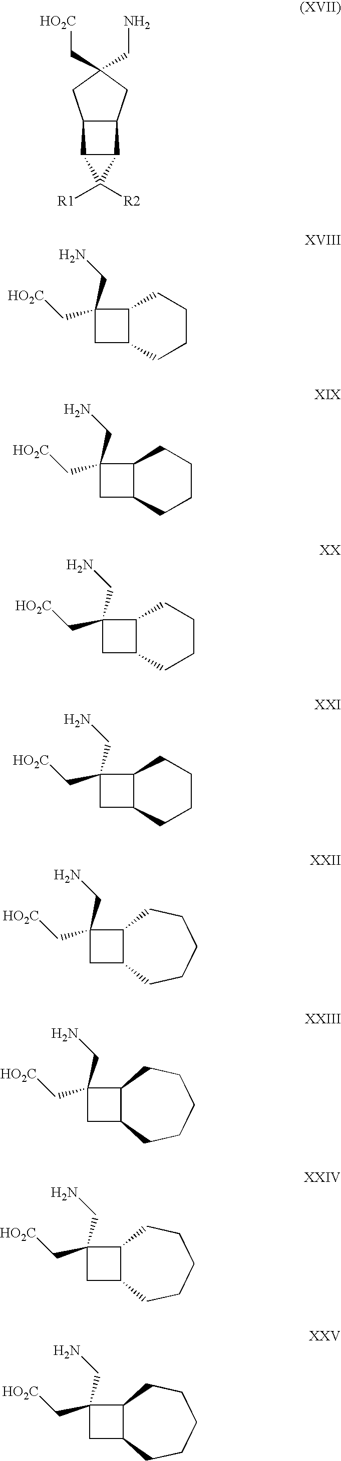

wherein R¹ and R² are each independently selected from H, straight or branched alkyl of 1-6 carbon atoms, cycloalkyl of from 3-6 carbon atoms, phenyl and benzyl, subject to the proviso that, except in the case of a tricyclooctane compound of formula (XVII), R¹ and R² are not simultaneously hydrogen; for use in the treatment of a number of indications, including pain.

International Patent Application Publication No. WO-A-03/082807, describes compounds of the formula I, below:

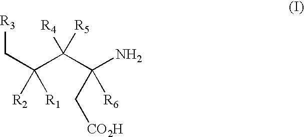

wherein R₁ is hydrogen or (C₁-C₆)alkyl optionally substituted with from one to five fluorine atoms;
R₂ is hydrogen or (C₁-C₆)alkyl optionally substituted with from one to five fluorine atoms; or
R₁ and R₂, together with the carbon to which they are attached, form a three to six membered cycloalkyl ring;
R₃ is (C₁-C₆)alkyl, (C₃-C₆)cycloalkyl, (C₃-C₆)cycloalkyl-(C₁-C₃)alkyl, phenyl, phenyl-(C₁-C₃)alkyl, pyridyl, pyridyl-(C₁-C₃)alkyl, phenyl-N(H)—, or pyridyl-N(H)—, wherein each of the foregoing alkyl moieties can be optionally substituted with from one to five fluorine atoms, preferably with from zero to three fluorine atoms, and wherein said phenyl and said pyridyl and the phenyl and pyridyl moieties of said phenyl-(C₁-C₃)alkyl and said pyridyl-(C₁-C₃)alkyl, respectively, can be optionally substituted with from one to three substituents, preferably with from zero to two substituents, independently selected from chloro, fluoro, amino, nitro, cyano, (C₁-C₃)alkylamino, (C₁-C₃)alkyl optionally substituted with from one to three fluorine atoms and (C₁-C₃)alkoxy optionally substituted with from one to three fluorine atoms;
R₄ is hydrogen or (C₁-C₆)alkyl optionally substituted with from one to five fluorine atoms;
R₅ is hydrogen or (C₁-C₆)alkyl optionally substituted with from one to five fluorine atoms; and
R₆ is hydrogen or (C₁-C₆)alkyl;
or a pharmaceutically acceptable salt thereof.

International Patent Application Publication No. WO-A-04/039367 describes compounds of the formula (I), below:

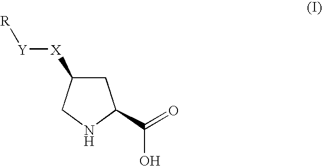

wherein
either X is O, S, NH or CH₂ and Y is CH₂ or a direct bond, or Y is O, S or NH and X is CH₂; and
R is a 3-12 membered cycloalkyl, 4-12 membered heterocycloalkyl, aryl or heteroaryl, where any ring may be optionally substituted with one or more substituents independently selected from halogen, hydroxy, cyano, nitro, amino, hydroxycarbonyl,
C₁-C₆ alkyl, C₁-C₆ alkenyl, C₁-C₆ alkynyl,
C₁-C₆ alkoxy, hydroxyC₁-C₆ alkyl, C₁-C₆ alkoxyC₁-C₆ alkyl, perfluoro C₁-C₆ alkyl, perfluoroC₁-C₆ alkoxy,
C₁-C₆ alkylamino, di-C₁-C₆ alkylamino, aminoC₁-C₆ alkyl, C₁-C₆ alkylaminoC₁-C₆ alkyl, di-C₁-C₆ alkylaminoC₁-C₆ alkyl,
C₁-C₆acyl, C₁-C₆acyloxy, C₁-C₆acyloxyC₁-C₆ alkyl, C₁-C₆ acylamino,
C₁-C₆ alkylthio, C₁-C₆ alkylthiocarbonyl, C₁-C₆ alkylthioxo, C₁-C₆ alkoxycarbonyl,
C₁-C₆ alkylsulfonyl, C₁-C₆ alkylsulfonylamino, aminosulfonyl, C₁-C₆ alkylaminosulfonyl, di-C₁-C₆ alkylaminosulfonyl,
3-8 membered cycloalkyl, 4-8 membered heterocycloalkyl, phenyl and monocyclic heteroaryl;
or a pharmaceutically acceptable salt thereof.

International Patent Application Publication No. WO-A-05/030700 describes compounds of formula (I) below:

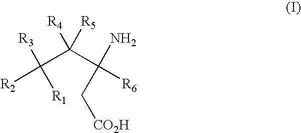

and their pharmaceutically acceptable salts, wherein
R₁ is a hydrogen atom or (C₁-C₆)alkyl optionally substituted with from one to five fluorine atoms;
R₂ is a hydrogen atom or (C₁-C₆)alkyl optionally substituted with from one to five fluorine atoms; or
R₁ and R₂, together with the carbon to which they are attached, form a three- to six-membered cycloalkyl ring;
R₃ is a hydrogen atom, (C₁-C₆)alkyl, (C₃-C₆)cycloalkyl, (C₃-C₆)cycloalkyl-(C₁-C₃)alkyl, phenyl, phenyl-(C₁-C₃)alkyl, pyridyl, or pyridyl-(C₁-C₃)alkyl, wherein the alkyl and cycloalkyl moieties or substituents are optionally substituted with from one to five fluorine atoms, preferably with from zero to three fluorine atoms, and the phenyl and pyridyl substituents and the phenyl and pyridyl moieties of the phenyl-(C₁-C₃)alkyl and the pyridyl-(C₁-C₃)alkyl substituents are optionally substituted with from one to five substituents, preferably with from zero to two substituents, independently selected from chloro, fluoro, amino, nitro, cyano, hydroxy, (C₁-C₃)alkylamino, (C₁-C₃)alkyl optionally substituted with from one to three fluorine atoms, and (C₁-C₃)alkoxy optionally substituted with from one to three fluorine atoms;
R₄ is a hydrogen atom or (C₁-C₆)alkyl optionally substituted with from one to five fluorine atoms;
R₅ is a hydrogen atom or (C₁-C₆)alkyl optionally substituted with from one to five fluorine atoms;
R₄ and R₅, together with the carbon to which they are attached, form a three- to six-membered cycloalkyl ring; and
R₆ is a hydrogen atom or (C₁-C₆)alkyl;
with the proviso that R₁, R₂, R₃, R₄, R₅, and R₆ are not simultaneously hydrogen atoms.

Further specific alpha-2-delta ligands include gabapentin, pregabalin, 3-methylgabapentin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-fluoro-phenoxymethyl)-pyrrolidine-2-carboxylic acid, (2S,4S)-4-(2,3-difluorobenzyl)-pyrrolidine-2-carboxylic acid, (2S,4S)-4-(3-fluorobenzyl)proline, (2S,4S)-4-(3-chlorophenoxy)proline, (3R,4R,5R)-3-amino-4,5-dimethyl-heptanoic acid and (3R,4R,5R)-3-amino-4,5-dimethyl-octanoic acid, or pharmaceutically acceptable salts thereof.

Gabapentin has been successfully applied in the treatment of pain in a large number of patients. For example, M. Backonja et al., JAMA, 280, 1831-1836 (1998) reports the efficacy and safety of gabapentin in reducing pain attributed to the peripheral neuropathy of diabetes. Statistically significant scores were noted on both the primary efficacy measures as well as numerous secondary outcome measures of pain for those patients treated with gabapentin versus those receiving placebo. Approximately 60% of patients receiving gabapentin noted at least a moderate improvement (ie a score of 1 or 2) on the Patient Global Impression of Change (this is a scale of 1 to 7, wherein 1 represents a marked improvement in condition, 2 a moderate improvement, 3 a minimal improvement, 4 represents no change, 5 minimally worse, 6 moderately worse and 7 a marked deterioration in condition). See “A randomized, placebo-controlled trial”, R. H. Dworkin et al. Neurology 60, 1274-1283 (2003).

As can be seen from the above study, there remains a proportion of patients suffering from pain, especially neuropathic pain, that for reasons that are not understood do not respond to treatment with alpha-2-delta ligands such as gabapentin. For the purposes of the present invention ‘a patient who does not respond to treatment’ is defined as a patient noting a minimal improvement or worse on the Patient Global Impression of Change, ie a score of 3 or higher.

D. A. Simpson, Journal of Clinical Neuromuscular Disease, 3(2), 53-62 (2001) reports that gabapentin is efficacious in the treatment of painful diabetic neuropathy. The document further discloses that patients receiving gabapentin plus venlafaxine, which is a potent inhibitor of both serotonin and norepinephrine reuptake, as well as a weak inhibitor of dopamine reuptake, showed significant improvements in pain reduction, mood disturbance, and quality of life. The author concludes that additional treatment with venlafaxine is efficacious in patients who do not respond to gabapentin monotherapy.

We have surprisingly found that the highly selective norepinephrine reuptake inhibitor (S,S)-reboxetine is efficacious in treating pain in patients who do not respond to treatment with alpha-2-delta ligands such as gabapentin. This is contrary to what would have been expected, as based on the teaching of the prior art, it would have been thought necessary for a compound to possess both serotonin and norepinephrine reuptake inhibitory activity in order for it to be efficacious in the treatment of pain in gabapentin non-responders.

We have also surprisingly found that (S,S)- and racemic reboxetine display efficacy as a mental performance and mood enhancer.

### SUMMARY OF THE INVENTION

According to a first aspect of the invention, there is provided the use of (S,S)- or racemic reboxetine, or a pharmaceutically acceptable salt thereof, in the manufacture of a medicament for the treatment of a pain condition selected from:

neuropathic pain (defined as pain caused by lesion or dysfunction of the nervous system, including diabetic neuropathy, post herpetic neuralgia, cancer neuropathy, HIV neuropathy, trigeminal neuralgia, phantom limb pain, carpal tunnel syndrome, neuropathic pain caused by chronic alcoholism, hypothyroidism, uremia, or vitamin deficiencies, central post-stroke pain, and pain caused by multiple sclerosis, spinal cord injury, Parkinson's disease and epilepsy),
nociceptive pain (including moderate to severe acute nociceptive pain), cancer pain (including tumour related pain, e.g. bone pain, headache and facial pain, viscera pain, pain associated with cancer therapy, e.g. postchemotherapy syndromes, chronic postsurgical pain syndromes, post radiation syndromes, and cancer related acute pain syndromes, such as those caused by therapeutic interactions such as chemotherapy toxicity, immunotherapy, hormonal therapy and radiotherapy), back pain (including back pain due to herniated or ruptured intervertebral discs or abnormalities of the lumbar facet joints, sacroiliac joints, paraspinal muscles or the posterior longitudinal ligament), inflammatory pain (including arthritic pain, rheumatoid disease, rheumatoid arthritis and symptomatic osteoarthritis),
musculo-skeletal disorders (including myalgia, spondylitis, sero-negative (non-rheumatoid) arthropathies, non-articular rheumatism, dystrophinopathy, glycogenolysis, polymyositis and pyomyositis),
visceral pain, such as heart and vascular pain (including pain caused by angina, myocardial infarction, mitral stenosis, pericarditis, Raynaud's phenomenon, scleredoma, scleredoma, and skeletal muscle ischemia), and pain caused by gastrointestinal disorders (including digestive visceral pain, non-digestive visceral pain, functional bowel disorders (FBD), inflammatory bowel diseases (IBD), gastro-esophageal reflux, dyspepsia, irritable bowel syndrome (IBS), functional abdominal pain syndrome (FAPS), Crohn's disease, ileitis, and ulcerative colitis, pain associated with dysmenorrhea, pelvic pain, cystitis and pancreatitis), pain from strains/sprains,
post-operative pain (pain following any type of surgical procedure), posttraumatic pain,
burns,
renal colic,
acute pain,
central nervous system trauma,
head pain (including cluster headache), and
orofacial pain (including dental pain and temporomandibular myofascial pain).

In a second aspect, the invention comprises a method for the treatment of a pain condition selected from:

neuropathic pain (defined as pain caused by lesion or dysfunction of the nervous system, including diabetic neuropathy, post herpetic neuralgia, cancer neuropathy, HIV neuropathy, trigeminal neuralgia, phantom limb pain, carpal tunnel syndrome, neuropathic pain caused by chronic alcoholism, hypothyroidism, uremia, or vitamin deficiencies, central post-stroke pain, and pain caused by multiple sclerosis, spinal cord injury, Parkinson's disease and epilepsy),
nociceptive pain (including moderate to severe acute nociceptive pain), cancer pain (including tumour related pain, e.g. bone pain, headache and facial pain, viscera pain, pain associated with cancer therapy, e.g. postchemotherapy syndromes, chronic postsurgical pain syndromes, post radiation syndromes, and cancer related acute pain syndromes, such as those caused by therapeutic interactions such as chemotherapy toxicity, immunotherapy, hormonal therapy and radiotherapy),
back pain (including back pain due to herniated or ruptured intervertebral discs or abnormalities of the lumbar facet joints, sacroiliac joints, paraspinal muscles or the posterior longitudinal ligament), inflammatory pain (including arthritic pain, rheumatoid disease, rheumatoid arthritis and symptomatic osteoarthritis),
musculo-skeletal disorders (including myalgia, spondylitis, sero-negative (non-rheumatoid) arthropathies, non-articular rheumatism, dystrophinopathy, glycogenolysis, polymyositis and pyomyositis),
visceral pain, such as heart and vascular pain (including pain caused by angina, myocardial infarction, mitral stenosis, pericarditis, Raynaud's phenomenon, scleredoma, scleredoma, and skeletal muscle ischemia), and pain caused by gastrointestinal disorders (including digestive visceral pain, non-digestive visceral pain, functional bowel disorders (FBD), inflammatory bowel diseases (IBD), gastro-esophageal reflux, dyspepsia, irritable bowel syndrome (IBS), functional abdominal pain syndrome (FAPS), Crohn's disease, ileitis, and ulcerative colitis, pain associated with dysmenorrhea, pelvic pain, cystitis and pancreatitis),
pain from strains/sprains,
post-operative pain (pain following any type of surgical procedure),
posttraumatic pain,
burns,
renal colic,
acute pain,
central nervous system trauma,
head pain (including cluster headache), and
orofacial pain (including dental pain and temporomandibular myofascial pain), in a mammal, including a human, the method comprising administering to the mammal an effective amount of (S,S)- or racemic reboxetine, or a pharmaceutically acceptable salt thereof.

The invention provides in a third aspect the use of optically pure (S,S)-reboxetine, or a pharmaceutically acceptable salt thereof, said compound being substantially free of (R,R)-reboxetine, in the manufacture of a medicament for the treatment of pain in a patient refractory to an alpha-2-delta ligand.

The invention further provides in a fourth aspect a method for the treatment of pain in a patient refractory to an alpha-2-delta ligand, the method comprising administering to a patient in need of such treatment an effective amount of optically pure (S,S)-reboxetine, or a pharmaceutically acceptable salt thereof, said compound being substantially free of (R,R)-reboxetine.

The invention additionally provides in a fifth aspect the use of (S,S)- or racemic reboxetine or a pharmaceutically acceptable salt thereof in the manufacture of a medicament for use as a mental performance or mood enhancer.

The invention also provides in a sixth aspect a method for increasing mental performance or enhancing mood, the method comprising administering to a patient in need of such treatment an effective amount of (S,S)- or racemic reboxetine or a pharmaceutically acceptable salt thereof.

Other advantages and preferred aspects of the invention are described below.

### DETAILED DESCRIPTION OF PREFERRED EMBODIMENTS

The uses and methods of the present invention may employ optically pure (S,S)-reboxetine, or a pharmaceutically acceptable salt thereof, said compound being substantially free of (R,R)-reboxetine. The phrases ‘optically pure (S,S) reboxetine’ and ‘substantially free of (R,R) reboxetine,’ as used herein, mean that the composition contains a greater proportion of (S,S) reboxetine in relation to (R,R) reboxetine. In a preferred embodiment, the phrases mean that the composition is at least 90 percent by weight (wt. %) of (S,S) reboxetine, and 10 wt. % or less of (R,R) reboxetine. In a more preferred embodiment the phrases mean that the composition contains at least 97 wt. % of (S,S) reboxetine, and 3 wt. % or less of (R,R) reboxetine. In an even more preferred embodiment, the phrases mean that the composition contains at least 99 wt. % of (S,S) reboxetine, and 1 wt. % or less of (R,R) reboxetine. In a most preferred embodiment, the phrases ‘optically pure (S,S) reboxetine’ and ‘substantially free of its (R,R) stereoisomer,’ as used herein, mean that the composition contains greater than 99 wt. % of (S,S) reboxetine. The foregoing percentages are based upon the total amount of reboxetine present in the composition.

The first and second aspects of the present invention comprises the use of (S,S)- or racemic reboxetine or a pharmaceutically acceptable salt thereof in the manufacture of a medicament for the treatment of a pain condition selected from those listed above.

It will be appreciated that the term “treatment” as used herein refers to curative, prophylactic and palliative treatment.

(S,S)- and racemic reboxetine contain a basic group, and may therefore be converted to a pharmaceutically acceptable salt by reaction with an acid.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Preferred examples of salts include the mesylate, fumarate and succinate salts, and the succinate is especially preferred.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of (S,S)- or racemic reboxetine may be readily prepared in a conventional manner by mixing together solutions of (S,S)- or racemic reboxetine and the desired acid, as appropriate. The salt may precipitate from solution and be collected by filtration or may be recovered by evaporation of the solvent. The degree of ionisation in the salt may vary from completely ionised to almost non-ionised.

(S,S)- or racemic reboxetine may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

(S,S)- or racemic reboxetine may be administered alone or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of (S,S)- or racemic reboxetine and methods for their preparation will be readily apparent to those skilled in the art. Such compositions and methods for their preparation may be found, for example, in ‘Remington's Pharmaceutical Sciences’, 19th Edition (Mack Publishing Company, 1995).

According to the invention, (S,S)- or racemic reboxetine may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

(S,S)- or racemic reboxetine may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al., Pharmaceutical Technology On-line, 25(2), 1-14 (2001). The use of chewing gum to achieve controlled release is described in WO 00/35298.

(S,S)- or racemic reboxetine may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of (S,S)- or racemic reboxetine used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

(S,S)- or racemic reboxetine may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

(S,S)- or racemic reboxetine can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of (S,S)- or racemic reboxetine comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying. Capsules (made, for example, from gelatin or HPMC), blisters and cartridges for use in an inhaler or insufflator may be formulated to contain a powder mix of (S,S)- or racemic reboxetine, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of (S,S)- or racemic reboxetine per actuation and the actuation volume may vary from 1 μl to 100 μl. A typical formulation may comprise (S,S)- or racemic reboxetine, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid) (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff”.

(S,S)- or racemic reboxetine may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

Desirably, daily dose of the composition (e.g., tablet, sachet, or capsule) contains from about 0.1 to about 10 mg of (S,S)- or racemic reboxetine. More preferably, each dose of the composite contains about 0.5 to about 10 mg of the active ingredient, (S,S)- or racemic reboxetine. This dosage form permits the full daily dosage of about 0.5 to about 2.5 mg to be administered in one or two oral doses. This will allow for tablets containing 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9. 2.0, 2.1, 2.2, 2.3, 2.4, or 2.5 mg of (S,S)- or racemic reboxetine.

According to the present invention, (S,S)-reboxetine is useful for the treatment of pain in a patient refractory to an alpha-2-delta ligand. For the purposes of the present invention a ‘patient refractory to an alpha-2-delta ligand’ is defined as a patient noting a minimal improvement or worse on the Patient Global Impression of Change, ie a score of 3 or worse, when treated with an alpha-2-delta ligand.

In one embodiment, the alpha-2-delta ligand is gabapentin or a pharmaceutically acceptable salt thereof. Therefore, in a preferred embodiment, the invention comprises the use of optically pure (S,S)-reboxetine, or a pharmaceutically acceptable salt thereof, said compound being substantially free of (R,R)-reboxetine, in the manufacture of a medicament for the treatment of pain in a patient who does not respond to gabapentin or a pharmaceutically acceptable salt thereof (in other words, a patient who notes a minimal improvement or worse on the Patient Global Impression of Change, ie a score of 3 or worse, when treated with gabapentin or a pharmaceutically acceptable salt thereof).

In another embodiment, the alpha-2-delta ligand is pregabalin or a pharmaceutically acceptable salt thereof. Therefore, in a preferred embodiment, the invention comprises the use of optically pure (S,S)-reboxetine, or a pharmaceutically acceptable salt thereof, said compound being substantially free of (R,R)-reboxetine, in the manufacture of a medicament for the treatment of pain in a patient who does not respond to pregabalin or a pharmaceutically acceptable salt thereof (in other words, a patient who notes a minimal improvement or worse on the Patient Global Impression of Change, ie a score of 3 or worse, when treated with pregabalin or a pharmaceutically acceptable salt thereof).

In another embodiment, the alpha-2-delta ligand is [(1R,5R,6S)-6-(aminomethyl)bicyclo-[3.2.0]hept-6-yl]acetic acid or a pharmaceutically acceptable salt thereof. Therefore, in a preferred embodiment, the invention comprises the use of optically pure (S,S)-reboxetine, or a pharmaceutically acceptable salt thereof, said compound being substantially free of (R,R)-reboxetine, in the manufacture of a medicament for the treatment of pain in a patient who does not respond to [(1R,5R,6S)-6-(aminomethyl)bicyclo-[3.2.0]hept-6-yl]acetic acid or a pharmaceutically acceptable salt thereof (in other words, a patient who notes a minimal improvement or worse on the Patient Global Impression of Change, ie a score of 3 or worse, when treated with [(1R,5R,6S)-6-(aminomethyl)bicyclo-[3.2.0]hept-6-yl]acetic acid or a pharmaceutically acceptable salt thereof).

In another embodiment, the alpha-2-delta ligand is (1α,3α,5α)(3-amino-methyl-bicyclo[3.2.0]hept-3-yl)-acetic acid or a pharmaceutically acceptable salt thereof. Therefore, in a preferred embodiment, the invention comprises the use of optically pure (S,S)-reboxetine, or a pharmaceutically acceptable salt thereof, said compound being substantially free of (R,R)-reboxetine, in the manufacture of a medicament for the treatment of pain in a patient who does not respond to (1α,3α,5α)(3-amino-methyl-bicyclo[3.2.0]hept-3-yl)-acetic acid or a pharmaceutically acceptable salt thereof (in other words, a patient who notes a minimal improvement or worse on the Patient Global Impression of Change, ie a score of 3 or worse, when treated with (1α,3α,5α)(3-amino-methyl-bicyclo[3.2.0]hept-3-yl)-acetic acid or a pharmaceutically acceptable salt thereof).

In another embodiment, the alpha-2-delta ligand is (2S,4S)-4-(3-chlorophenoxy)proline or a pharmaceutically acceptable salt thereof. Therefore, in a preferred embodiment, the invention comprises the use of optically pure (S,S)-reboxetine, or a pharmaceutically acceptable salt thereof, said compound being substantially free of (R,R)-reboxetine, in the manufacture of a medicament for the treatment of pain in a patient who does not respond to (2S,4S)-4-(3-chlorophenoxy)proline or a pharmaceutically acceptable salt thereof (in other words, a patient who notes a minimal improvement or worse on the Patient Global Impression of Change, ie a score of 3 or worse, when treated with (2S,4S)-4-(3-chlorophenoxy)proline or a pharmaceutically acceptable salt thereof).

In another embodiment, the alpha-2-delta ligand is (2S,4S)-4-(3-fluorobenzyl)proline or a pharmaceutically acceptable salt thereof. Therefore, in a preferred embodiment, the invention comprises the use of optically pure (S,S)-rebooxetine, or a pharmaceutically acceptable salt thereof, said compound being substantially free of (R,R)-reboxetine, in the manufacture of a medicament for the treatment of pain in a patient who does not respond to (2S,4S)-4-(3-fluorobenzyl)proline or a pharmaceutically acceptable salt thereof (in other words, a patient who notes a minimal improvement or worse on the Patient Global Impression of Change, ie a score of 3 or worse, when treated with (2S,4S)-4-(3-fluorobenzyl)proline or a pharmaceutically acceptable salt thereof).

(S,S)- or racemic reboxetine may be used according to the present invention for the treatment of neuropathic pain. The activity of (S,S)- and racemic reboxetine in the treatment of neuropathic pain may be measured according to the following test protocol.

Animals: Male Sprague Dawley rats (200-250 g), obtained from Charles River, (Margate, Kent, U.K.) are housed in groups of 6. All animals are kept under a 12 h light/dark cycle (lights on at 07 h 00 min) with food and water ad libitum. All experiments are carried out by an observer unaware of drug treatments.

### CCl Surgery in the Rat

Animals are anaesthetised with isoflurane. The sciatic nerve is ligated as previously described by Bennett and Xie, 1988. Animals are placed on a homeothermic blanket for the duration of the procedure. After surgical preparation the common sciatic nerve is exposed at the middle of the thigh by blunt dissection through biceps femoris. Proximal to the sciatic trifurcation, about 7 mm of nerve is freed of adhering tissue and 4 ligatures (4-0 silk) are tied loosely around it with about 1 mm spacing. The incision is closed in layers and the wound treated with topical antibiotics.

### Evaluation of Allodynia

Static allodynia is measured using Semmes-Weinstein von Frey hairs (Stoelting, Ill., U.S.A.). Animals are placed into wire mesh bottom cages allowing access to the underside of their paws. Animals are habituated to this environment prior to the start of the experiment. Static allodynia is tested by touching the plantar surface of the animals right hind paw with von Frey hairs in ascending order of force (0.7, 1.2, 1.5, 2, 3.6, 5.5, 8.5, 11.8, 15.1 and 29 g) for up to 6 sec. Once a withdrawal response is established, the paw is re-tested, starting with the next descending von Frey hair until no response occurred. The highest force of 29 g lifted the paw as well as eliciting a response, thus represented the cut off point. The lowest amount of force required to elicit a response is recorded as the PWT in grams.

Dynamic allodynia is assessed by lightly stroking the plantar surface of the hind paw with a cotton bud. Care is taken to perform this procedure in fully habituated rats that are not active to avoid recording general motor activity. At least three measurements are taken at each time point the mean of which represented the paw withdrawal latency (PWL). If no reaction is exhibited within 15 s the procedure is terminated and animals are assigned this withdrawal time. Thus 15 s effectively represents no withdrawal. A withdrawal response is often accompanied with repeated flinching or licking of the paw. Dynamic allodynia is considered to be present if animals responded to the cotton stimulus before 8 s of stroking.

(S,S)- and racemic reboxetine may also be used according to the present invention for the treatment of nociceptive pain. The activity of (S,S)- and racemic reboxetine in the treatment of nociceptive pain may be measured according to the following test protocols.

### Hotplate

Experimental Procedure: Charles River male Sprague Dawley rats (75-200 g) are placed on a hot plate (Ugo Basile, Italy) maintained at 55±5° C. The time between placement of the animal on the hot plate and occurrence of either licking of fore or hind paw, shaking or jumping off the surface is measured. Baseline measurements will be made and animals reassessed following drug administration. The cut off time for hot plate latencies is set at 20 seconds to prevent tissue damage.

### Ovariohysterectomy (OVX)

Experimental Procedure: Charles River female Sprague Dawley rats (175-200 g) are placed into an anaesthetic chamber and anaesthetised with a 2% isofluorane O₂ mixture. During surgery, anaesthesia is maintained via a nose cone. OVX is performed via a midline incision (2 cm in length) in the linea alba, whilst the animal is on a heat blanket. The ovarian ligaments and cervix are ligated with 5-0 silk, using a single clamp technique. The ovaries and uterus are then removed. The abdominal wall is closed using 4 simple interrupted sutures and the skin closed using 4 wound clips. Immediately after surgery animals are placed in individual plexiglass chambers. Once the animal has recovered from the anaesthetic the abdominal body postures are recorded in 30 min bins at various time points. Postures scored are humpback position, contraction of the muscle of the abdomen associated with inward movements of the hind limb, stretching of the body and squashing of the lower abdomen against the floor. Each of these behaviours is scored as one posture.

### Brennan

Experimental Procedure: Charles River male Sprague Dawley rats (125-250 g) are placed into an anaesthetic chamber and anaesthetised with a 2% isofluorane O₂ mixture. During surgery, anaesthesia is maintained via a nose cone. The plantar aspect of the right hind paw is cleaned with 50% ethanol. A 1 cm long longitudinal incision is made with a number 11 blade through the skin and fascia of the plantar aspect of the foot, starting 0.5 cm from the proximal edge of the heel and extending toward the toes. The plantaris muscle is elevated using forceps and incised longitudinally, the muscle origin and insertion remain intact. After haemostasis with gentle pressure, the skin is closed with two simple sutures of braided silk.

### Behavioural Endpoint Measures

### Punctate Allodynia

Experimental procedure: Animals are habituated to wire bottom test cages prior to the assessment of allodynia. Punctate allodynia is evaluated by application of von Frey hairs (Stoelting, Wood Dale, Ill., U.S.A.) in ascending order of force (0.6, 1, 1.4, 2, 4, 6, 8, 10, 15 and 26 grams) to the plantar surface of hind paws. Each von Frey hair is applied to the paw for a maximum of 6 seconds, or until a withdrawal response occurs. Once a withdrawal response to a von Frey hair is established, the paw is re-tested, starting with the filament below the one that produced a withdrawal, and subsequently with the remaining filaments in descending force sequence until no withdrawal occurs. The highest force of 26 g lifts the paw as well as eliciting a response, thus representing the cut off point. Each animal has both hind paws tested in this manner. The lowest amount of force required to elicit a response is recorded as paw withdrawal threshold (PWT) in grams. Static allodynia is defined as present if animals respond to a stimulus of, or less than, 4 g, which is innocuous in normal rats.

### Dynamic Allodynia

Experimental procedure: Dynamic allodynia is assessed by lightly stroking the plantar surface of the hind paw with a cotton bud. Care is taken to perform this procedure in fully habituated rats that are not active, to avoid recording general motor activity. At least two measurements are taken at each time point, the mean of which represents the paw withdrawal latency (PWL). If no reaction is exhibited within 15 s the procedure is terminated and animals assigned this withdrawal time. Thus, 15 s effectively represents no withdrawal. A withdrawal response is often accompanied with repeated flinching or licking of the paw. Dynamic allodynia is considered to be present if animals responded to the cotton stimulus within 8 s of commencing stroking.

### Radiant Heat Paw Withdrawal

Experimental procedure: Thermal paw withdrawal is assessed using the rat plantar test (Ugo Basile, Italy) following a modified method of Hargreaves et al., 1988. Rats are habituated to the apparatus that consists of three individual perspex boxes on an elevated glass table. A mobile radiant heat source is located under the table and focused onto the hind paw and paw withdrawal latencies (PWL) are recorded. There is an automatic cut off point of 22.5 s to prevent tissue damage. PWL are taken 2-3 times for both hind paws of each animal, the mean of which represents baselines for right and left hind paws. The apparatus is calibrated to give a PWL of approximately 10 s.

### Weight Bearing

Experimental procedure: Animals are examined for hypersensitivity in the weight-bearing test, using an “incapacitance tester” (Linton Instruments, Diss, Norfolk, U.K.). Rats were positioned with their fore limbs up on a perspex slope and hind limb weight distribution was measured via force transducers under each of the hind paws. Each animal is placed in the apparatus and the weight load exerted by the hind paws is noted. The difference in weight bearing is calculated by subtracting the ipsilateral (injured) paw from the contralateral paw (normal) and this constitutes the raw data.

(S,S)- and racemic reboxetine may also be used according to the present invention for the treatment of inflammatory pain. The activity of (S,S)- and racemic reboxetine in the treatment of inflammatory pain may be measured according to the following test protocol.

### CFA-Induced Weight Bearing Deficits in Rats

Male 7-week-old SD rats are fasted overnight. CFA (300 μg of Mycobacterium Tuberculosis H37 RA (Difco Laboratories) in 100 μL of liquid paraffin (Wako)) is injected into the rat's right hind footpad. Two days after the administration of CFA, the changes in hind paw weight distribution between the left (ipsilateral) and the right (contralateral) limbs are measured as an index of pain by using Linton Incapacitance tester (Linton Instrumentation, UK). The test compound suspended in 0.1% MC (Wako) is administered orally in a volume of 1 mL per 100 g body weight. Each animal is placed in the apparatus and the weight load exerted by the hind paws is measured before, 1, 2 and 4 hours after drug administration.

### Carrageenin-Induced Mechanical Hyperalgesia in Rats

Male 4-week-old SD rats are fasted overnight. Hyperalgesia is induced by intraplantar injection of λ^{~-carrageenin (}0.1 ml of 1% w/v solution in saline, Zushikagaku). The test compound (1 ml of 0.1% methylcellulose/100 g body weight) is given orally at 5.5 hours after the carrageenin injection. The paw withdrawal threshold (gram) is measured by analgesimeter (Ugo Basile) at 3.5, 4.5, 6.5 and 7.5 hours after the carrageenin injection. (Randall L. O. & Selitto I. J., Arch. Int. Pharmacodyn. 111, 409-419, 1957)

### Carrageenin-Induced Thermal Hyperalgesia in Rats

Male 4-week-old SD rats are fasted overnight. Hyperalgesia is induced by intraplantar injection of λ^{~-carrageenin (}0.1 ml of 1% w/v solution in saline, Zushikagaku) into the right hindpaw of the rats. The test compound (1 ml of 0.1% methylcellulose/100 g body weight) is given orally at 5 hours after the carrageenin injection. Rats are placed in plastic cages of plantar test apparatus (Ugo Basile) and the mobile radiant heat source is focused on right hind paw of the rats. The thermal paw-withdrawal latency (sec.) is measured before and 3, 4, 6 and 7 hours after the carrageenin injection.

(S,S)- and racemic reboxetine may also be used according to the present invention for the treatment of visceral pain. The activity of (S,S)- and racemic reboxetine in the treatment of visceral pain may be measured according to the following test protocols.

Several models are available to determine if (S,S)- and racemic reboxetine are effective in treating disorders of the viscera. These models include a LPS model (Eutamene H et al, J Pharmacol Exp Ther 2000 295 (1):162-7), a TNBS model (Diop L. et al, Gastroenterology 1999, 116, 4(2): A986), a IBD model (Clemett D, Markham A, Drugs 2000 April; 59(4):929-56), a pancreatic pain model (Isla A M, Hosp Med 2000 June; 61(6):386-9) and a visceral non digestive pain model (Boucher M et al, J Urol 2000 July; 164(1):203-8).

### TNBS-Induced Chronic Visceral Allodynia in Rats

In this experimental model of colonic distension in awake rats, previous injection of trinitrobenzenesulfonic acid (TNBS) into the proximal colon lowered the visceral pain threshold.

Materials and methods: Male Sprague-Dawley rats weighing 340-400 g are used. The animals are housed 3 per cage in a regulated environment (20±1° C., 50±5% humidity, with light 8:00 am to 8:00 pm). At day 0, under anesthesia (ketamine 80 mg/kg ip.; acepromazine 12 mg/kg Lp.), the injection of TNBS (50 mg/kg in ethanol 30%), or saline (1.5 ml/kg) for control rats, is performed into the proximal colon wall (1 cm from the cecum). After the surgery, animals are individually housed in polypropylene cages and kept in a regulated environment (20±1° C., 50±5% humidity, with light 8:00 a.m. to 8:00 p.m.) during 7 days. At day 7 after TNBS administration, a balloon (5-6 cm length) is inserted by anus, and kept in position (tip of balloon 5 cm from the anus) by taping the catheter to the base of the tail. Oral administration of the test compound is performed 1 h before the colonic distension cycle: the balloon is progressively inflated by steps of 5 mm Hg (0.667 kPa), from 0 to 75 mm Hg, each step of inflation lasting 30 s. Each cycle of colonic distension is controlled by a standard barostat. The threshold (mm Hg) corresponds to the pressure which produced the first abdominal contraction, and the cycle of distension is then discontinued. The colonic threshold is determined after performance of four cycles of distension on the same animal.

### LPS-Induced Rectal Hypersensitivity in Rats

Intraperitoneal injection of bacterial lipo-polysaccharide (LPS) has been shown to induce rectal hyperalgesia in awake rats.

Materials and methods: Animals are surgically prepared for electromyography: rats are anaesthetized by intraperitoneal injection of acepromazine (0.6 mg/kg) and ketamine (120 mg/kg). Three groups of three electrodes are implanted in the abdominal external oblique musculature, just superior to the inguinal ligament. Electrodes are exteriorized on the back of the neck and protected by a glass tube attached to the skin. Animals are individually housed in polypropylene cages and kept in a temperature-controlled room (21° C.). Food (UAR pellets, Epinay, France) and water are provided ad libitum.

Electromyographic recordings begin five days after surgery. The electrical activity of abdominal striated muscles is recorded with an electro-encephalograph machine (Mini VIII Alvar, Paris, France) using a short time constant (0.03 s) to remove low-frequency signals (<3 Hz) and a paper speed of 3.6 cm/min. Spike bursts are recorded as an index of abdominal contractions.

Distension procedure: Rats are placed in plastic tunnels (6 cm diameter×25 cm long), where they cannot move, escape, or turn around, in order to prevent damage to the balloon. Animals are accustomed to this procedure for four days before rectal distension in order to minimize stress reactions during experiments. The balloon used for distension is an arterial embolectomy catheter (Fogarty, Edwards Laboratories Inc.). Rectal distension is performed by insertion of the balloon (2 mm diameter×2 cm long) into the rectum, at 1 cm from the anus, and catheter is fixed at the base of the tail. It is inflated progressively with tepid water by steps of 0.4 ml, from 0 to 1.2 ml, each step of inflation lasting 5 min. To detect possible leakage, the volume of water introduced in the balloon is checked by complete removal with a syringe at the end of the distension period.

Experimental protocol: Rats are injected i.p. with LPS (1 mg/kg (Escherichia coli, serotype O111:B4) Sigma-Aldrich Chemical Co., St Louis, Mo.) or its vehicle, and rectal distension with concomitant electromyographic recording of abdominal contractions is performed 9 and 12 h after this administration. To determine the antinociceptive properties of (S,S)- or racemic reboxetine in hyperalgesia conditions, the test compound or the vehicle (NaCl 0.9% 0.3 ml/rat) are administered per os 1 h before rectal distension but preceded (12 h) by injection of LPS (1 mg/kg i.p.).

Drugs: All compounds are dissolved in sterile NaCl (0.9% isotonic saline) immediately before use.

### EXAMPLE

The following study is carried out to assess the efficacy of (S,S)-reboxetine in the treatment of patients with post-herpetic neuralgia (PHN) who fail to respond to gabapentin (GBP) treatment.

Study Population Male or female patients aged 18 years or older, with pain present more than 3 months after healing of the Herpes Zoster skin rash and who are also GBP treatment failures. No upper limit on the duration of PHN is imposed. GBP failures are defined as either:

Those patients who show no or minimal improvement on the Patient Global Impression of Change (PGIC) after treatment with GBP (1800 mg/day) administered for a period of 2 weeks

Or

Those who are unable to tolerate doses of GBP less than or equal to 1800 mg/day.

Study Design Randomized, double-blind, placebo-controlled, 2-treatment (placebo and active), age-stratified study in patients aged 18 years or older, who suffer from PHN. The study is comprised of 3 phases: (i) 7-weeks screening including 4-weeks GBP treatment, (ii) 5-weeks randomized treatment, and (iii) 1-week follow-up period.

All patients who meet entry criteria initially enter a 1-week run-in period during which a daily pain diary will be maintained. Thereafter, if the severity of pain meets relevant criteria for inclusion in the study, patients enter a 4-week period during which they will receive GBP treatment. Subsequently, the patients enter a 2-week washout period prior to randomization. Those patients who, at the end of the GBP treatment period, are identified as treatment failures and who meet randomization criteria after the two-week washout, are randomized to receive either-placebo or (S,S)-reboxetine treatment for a period of 5 weeks according to an age-stratified randomization.

The GBP dosing regimen is as shown in the table below.

| | Day | | | | | |
|---|---|---|---|---|---|---|
| | 1 | 2 | 3-6 | 7-9 | 10-12 | 13-28 |
| Mg/day | 300 | 600 | 900 | 1200 | 1500 | 1800 |
| Regimen | OD | BID | TID | TID | TID | TID |

OD = once daily;
BID = twice daily;
TID = 3 times daily

Randomisation to (S,S)-reboxetine or placebo is age-stratified to those aged less than 75 (<75, non-elderly) or to those aged 75 or more (≧75, elderly). These 2 age-stratified groups receive treatment medication in the following manner.

| | Day (Treatment) | | | | |
|---|---|---|---|---|---|
| | 1-7 | 8-14 | 15-21 | 22-28 | 29-35 |
| Non-elderly | 2 | 4 | 6 | 4 or 6 | 4 or 6 |
| dose | | | | | |
| (mg/day) | | | | | |
| Elderly dose | 1 | 2 | 3 | 2, 3 or 4 | 2, 3 or 4 |
| mg/day | | | | | |
| Regimen | OD | OD | OD | OD | OD |

### Non-Elderly Patients

Non-elderly patients characterized by good tolerability at 6 mg/day are maintained at this dose for the remaining period of double-blind treatment. In cases of poor tolerability in these patients, there is a provision for dose reduction at the end of treatment week 3 and week 4 from 6 mg/day to 4 mg/day. This is the only permissible dose reduction in the study.

### Elderly Patients

For patients characterized by good tolerability at 3 mg/day, the dose is routinely increased to 4 mg/day at the end of week 3 (V6). In case of poor tolerability, a single step dose reduction is allowed in the following manner: (i) at the end of week 3, from 3 to 2 mg/day, (ii) at the end of week 4, from either 4 to 3 mg/day or from 3 mg/day to 2 mg/day. Therefore, patients receive either 2, 3 or 4 mg/day.

Endpoints

| | |
|---|---|
| Primary | Change from baseline in weekly-average pain score. Pain |
| Endpoint(s): | intensity is based on the daily pain diary rating scale of |
| | 0-10. |
| Secondary | Responder rate (30% & 50% reduction in average pain |
| Endpoints: | score from baseline to end-point). |
| | Weekly average sleep interference scores from the daily |
| | sleep diary. |
| | SF-McGill pain questionnaire. |
| | Patient Global Impression of Change. |
| | Clinical Global Impression of Change. |
| | Patient Reported Outcomes |
| | SF-36. |
| | Hospital Anxiety and Depression Scale. |

1. (canceled)

2. A method according to claim 7, wherein the pain condition is neuropathic pain.

3. A method according to claim 7, wherein the pain condition is nociceptive pain.

4. A Method according to claim 7, wherein the pain condition is cancer pain.

5. A method according to claim 7, wherein the pain condition is inflammatory pain.

6. A method according to claim 7, wherein the pain condition is visceral pain.

7. A method for the treatment of a pain condition selected from neuropathic pain, nociceptive pain, cancer pain, back pain, inflammatory pain, musculo-skeletal disorders, visceral pain, pain from strains/sprains, post-operative pain, posttraumatic pain, burns, renal colic, acute pain, central nervous system trauma, head pain, and orofacial pain in a mammal, including a human, the method comprising administering to the mammal an effective amount of (S,S)- or racemic reboxetine, or a pharmaceutically acceptable salt thereof.

8. (canceled)

9. A method according to claim 12, wherein the pharmaceutically acceptable salt of (S,S)-reboxetine is the succinate.

10. A method according to claim 12, wherein the pain is neuropathic pain.

11. A method according to claim 12, wherein the neuropathic pain is post-herpetic neuralgia.

12. A method for the treatment of pain in a patient refractory to an alpha-2-delta ligand, the method comprising administering to a patient in need of such treatment an effective amount of optically pure (S,S)-reboxetine, or a pharmaceutically acceptable salt thereof, said compound being substantially free of (R,R)-reboxetine.

13. (canceled)

14. A method of increasing mental performance or enhancing mood, the method comprising administering to a patient in need thereof an effective amount of (S,S)- or racemic reboxetine or a pharmaceutically acceptable salt thereof.
